(19) United States
(12) Patent Application Publication
(10) Pub. No.: US 2011/0021355 A1
(43) Pub. Date: Jan. 27, 2011

(54) FORMULATION WITH REDUCED HYGROSCOPICITY

(75) Inventors: Jesko Zank, Idstein (DE); Martin Steinbeck, Köln (DE); Guido Becker, Krefeld (DE); Udo Van Stiphout, Moers (NL)

Correspondence Address: Briscoe, Kurt G.;Norris McLaughlin & Marcus, PA, 875 Third Avenue, 8th Floor, New York, NY, 10022, US

(73) Assignee: BAYER TECHNOLOGY SERVICES GMBH, Leverkusen (DE)

(21) Appl. No.: 12/934,286

(22) PCT Filed: Apr. 11, 2009
(86) PCT No.: PCT/EP09/02691

§ 371 Date: Sep. 24, 2010

(30) Foreign Application Priority Data
Apr. 24, 2008 (DE) 10 2008 020 701.2

(51) Int. Cl. A61K 9/00; A61K 33/08; A01N 59/06; A01N 59/00; A01N 25/08; A01P 3/00; A01P 5/00; A01P 7/00; A01P 13/00; A61P 1/04; A61P 7/06; A61P 19/10; A61P 23/00; A61P 5/00

(52) U.S. Cl. 504/119; 424/400; 424/688; 424/692; 424/693; 504/116.1

(57) ABSTRACT

The invention relates to formulations of hygroscopic solids with a lipophilic coating layer, and a method of production thereof.

The invention relates to formulations of hygroscopic solids with a lipophilic coating layer, and a method of production thereof.

A great many substances that are used for example as pharmaceutical active substances are hygroscopic. That is, the substances tend to absorb water, e.g. from the ambient air.

However, for some of these substances the absorption of water has an adverse effect on their properties, for example as pharmaceutical active substance. For example, there may be chemical decomposition of the active substance.

It is disclosed in WO 2007/142628 that one possibility for stabilization of these substances consists of providing a formulation of these substances that comprises at least one other substance whose affinity for absorption of water is even higher than that of the active substance, so that the substance that is to be present as active substance in the formulation is not exposed to moisture until the substance with higher affinity for absorption of water has reached a certain degree of saturation. Adsorbent resins, for instance, are disclosed as such substances that have higher affinity for absorption of water. In particular, WO 2007/142628 discloses copovidone (Plasdone®S-630) as a possible substance for absorption of water.

WO 2007/142628 further discloses a formulation comprising a moisture-sensitive active substance with at least one excipient, the excipient preferably being a binder. The method disclosed for production of this formulation comprises wet granulation of the active substance together with the binder. The resultant formulation thus comprises a matrix material of at least one excipient and the active substance.

The formulation and the method of production thereof have the disadvantage that penetration of moisture into the formulation is not prevented. Thus, the disclosure that stable formulations are obtained when, in the course of granulation, solvents with 95% ethanol are used instead of solvents with higher proportions of water, shows that there are narrow limits on the stabilization of the active substance in such formulations. It is to be assumed that the active substance is only stabilized for as long as the saturation of the at least one excipient with water does not reach an extent such that the affinity of the active substance for absorption of water becomes similar to that of the excipient.

EP 1 161 941 discloses a formulation of active substances and a method of production thereof, which envisages surface coating of powders of active substances. However, the desired technical effect relates to the possibility of improving the flowability of the powder as a result of the surface coating, to permit dry granulation or direct tableting of the dry substance.

The active substances that are suitable for such a formulation also comprise typical hygroscopic substances, such as calcium carbonate and magnesium oxide.

According to the disclosure, surface coating is by granulation of the coating material, also in powder form, with the active substance. The coating material can also comprise hygroscopic substances, such as calcium carbonate.

The disclosure in EP 1 161 941 is unfavourable with respect to the possibility of achieving stabilization of the active substance, because granulation of the active substance with the coating material is carried out. This leads to formation of agglomerates, which have porosity as an intrinsic property. As a result of this porosity, penetration of moisture into the core of the formulation cannot be prevented, so that reliable stabilization of the active substance is not possible.

Moreover, a very thick layer of coating material must be applied on the active substance, so as to achieve at least some inhibition of moisture penetration. This leads once again to smaller loadings of active substance per granule.

Based on the prior art, the object thus consists of developing formulations of hygroscopic solids and a method of production thereof, which overcome(s) the disadvantages of the prior art, so that the formulations of the hygroscopic solids are no longer hygroscopic.

It was found, surprisingly, that the object can be achieved with a formulation of at least one solid substance that is hygroscopic under normal conditions, which is characterized in that it comprises
1. a core, comprising at least one hygroscopic solid substance and
2. a lipophilic shell surrounding the core,
   the core and the shell being bound together by ionic interaction.

A substance is regarded as hygroscopic, in the sense of the invention, if in a period of 24 hours under normal conditions, it has a weight increase relative to its initial weight of at least 3%, through absorption of water from the moisture present in the surroundings. All substances that have a smaller weight increase under normal conditions are regarded as not hygroscopic in the context of the present invention.

Normal conditions mean, in the context of the present invention, a pressure of 1013 HPa, room temperature (20° C.), and a relative humidity of 100%.

The hygroscopic solids are usually active substances and/or fillers.

In the context of the present invention, active substances are usually substances that find pharmaceutical application or can be used in crop protection.

Active substances that find pharmaceutical application are for example active substances that can be used in the area of the treatment and alleviation of diseases of animals and of humans, for example agents for the treatment of acidosis, analeptics/antihypoxaemic agents, analgesics/antirheumatics, antacids, antiallergic agents, antianaemic agents, anti-arrhythmic agents, antibiotics/anti-infective agents, antidementia agents, antidiabetics, antidotes, antiepileptics, antihypertensives, antihyperglycaemic agents, antihypotensives, anticoagulants, antimycotics, antiparasitic agents, antiphlogistics, agents for treating arteriosclerosis, bronchodilators/antiasthmatics, cholagogues and agents for treating biliary tract disorders, cholinergic agents, corticoids, dermatic agents, diuretics, perfusion-promoting agents, agents for the treatment of addictions, enzyme inhibitors, fibrinolytics, geriatric agents, antipodagrics, gynaecologic remedies, hypatics, hypnotics/sedatives, immunomodulators, cardiac agents, coronary agents, laxatives, antilipaemic agents, local anaesthetics/neural therapeutic agents, gastrointestinal agents, migraine agents, muscle relaxants, ophthalmic agents, osteoporosis agents, otologic agents, psychoactive drugs, rhinologic agents, thyroid therapeutic agents, sex hormones, antispasmodic agents, alterants, urologic agents, vein therapeutic agents, vitamins and cytostatics.

Active substances that can be used in crop protection are, in the context of the present invention, substances from the classes of herbicides, fungicides, insecticides, acaricides, nematicides, bird repellents, plant nutrients and soil structure improving agents.

In the context of the present invention, fillers are all substances which, together with active substances, are permitted either for pharmaceutical use or correspondingly for use in a plant protection agent. Preferred fillers are the carbonate and phosphate salts and oxides and/or hydroxides of the alkali and alkaline-earth metals, for example calcium carbonate, calcium phosphate, calcium oxide, or cellulose derivatives, for example hydroxypropyl methylcellulose (HPMC), hydroxypropylcellulose (HPC) etc.

Active substances and/or fillers that can be used as antacids or gastrointestinal agents are preferred. Oxides and/or hydroxides of alkaline-earth metals are especially preferred. Calcium oxide and/or magnesium oxide are particularly preferred, and can be used both as fillers and as active substances for pharmaceutical use.

The present invention is not restricted, with respect to the hygroscopic solids, to the aforementioned preferred substances. Rather, these substances are often characterized in that even after prolonged storage they must not lose their properties as active substance through the absorption of water, so that the object of the present invention has especially positive effects here.

The invention is only restricted in that the hygroscopic solid substance must have at least one chemical group that permits the development of ionic interaction with the lipophilic shell.

The development of ionic interaction between the hygroscopic solid substance and the lipophilic shell is especially advantageous, because this results in orientation of the lipophilic substances with their lipophilic moiety outwards, so that even thin layers of the coating layer already achieve the positive technical effects of the coating. This is on the one hand economically advantageous, because less coating material has to be used, and on the other hand the properties of the hygroscopic solid substance, which are correlated with its particle size, are not affected to any great extent. For instance, the aerodynamic diameter of the formulation according to the invention stays almost the same, so that there is no consequent restriction of the application spectrum e.g. of inhalation products etc.

Preferably the hygroscopic solid substance comprises at least one positively charged chemical group.

The lipophilic shell surrounding the core according to the invention usually comprises a salt of the at least one hygroscopic solid substance with at least one organic acid, which is characterized by a lipophilic moiety and at least one acid group.

In the context of the present invention, a lipophilic moiety of an organic acid means that portion of an organic acid molecule that comprises at least four carbon atoms not arranged in the ring and optionally one or more multiple bonds between the at least four non-ring carbon atoms. Organic acids of the homologous series of the alkanes, alkenes and alkynes with at least four carbon atoms are preferred.

“Acid group” denotes, in the context of the present invention, that part of an organic acid molecule that leads, in an aqueous environment through dissociation of a proton (H*) that it contains, to a decrease of the aqueous medium. Acid groups of the carboxylates, sulphonates and phosphates are preferred.

The organic acids can possess one or more acid groups. Organic acids with only one acid group or mixtures of various organic acids with only one acid group are preferred.

Carboxylates of alkanes or alkenes with only one acid group and at least twelve carbon atoms or mixtures of various carboxylates of alkanes or alkenes with only one acid group are especially preferred.

Alkane-carboxy acids, e.g. capric acid, undecanoic acid, lauric acid, tridecanoic acid, myristic acid, pentadecanoic acid, palmitic acid, margaric acid, stearic acid, nonadecanoic acid and arachidic acid, or alkene-carboxy acids, for example linoleic acid, linolenic acid and arachidonic acid, or mixtures thereof, are quite especially preferred.

The formulations according to the invention are especially advantageous because they do not adversely affect the properties of the hygroscopic solid substance such as solubility in water and pH on dissolution in water. Furthermore, with the formulation according to the invention it is possible to achieve a rapid delayed release of the hygroscopic solid substance.

“Solubility in water” means, within the scope of the present invention, the maximum mass of the hygroscopic solid substance that can be dissolved molecularly in water at room temperature (20° C.) and atmospheric pressure (1013 HPa).

Preferably the solubility of the formulation decreases relative to the formulated substance by not more than 10%. Especially preferably, it does not change.

“pH on dissolution in water” means, within the scope of the present invention, the measurable pH after one hour in an aqueous solution with a concentration of 0.1 wt. % of a substance or formulation thereof.

Another object of the present invention is a method of production of formulations of hygroscopic solids that consist of a core of at least one hygroscopic solid substance and a lipophilic shell, characterized by the steps
a) preparation of a solution A of at least one acid in a solvent or solvent mixture 1;
b) mixing solution A with a hygroscopic solid substance, obtaining a dispersion B;
c) separating the solid fraction from dispersion B;
d) optionally post-treatment of the separated solid fraction from dispersion B from c).

The at least one organic acid of solution A in step a) according to the method of the invention comprises the organic acids, and mixtures thereof, already described in connection with the formulation according to the invention.

The solvent or solvent mixture 1 of solution A in step a) according to the method of the invention comprises nonpolar solvents or mixtures thereof with a polarity of max. 1.5 debye. Aniline, anisole and toluene, as well as mixtures thereof and mixtures with solvents of lower polarity, may be mentioned as non-exhaustive examples.

Solvents or solvent mixtures with a polarity of less than 0.5 debye are preferred. Hexane, cyclohexane, benzene, carbon disulphide, tetrachloroethylene, carbon tetrachloride or mixtures thereof with solvents of higher polarity may be mentioned as non-exhaustive examples.

Solvents or solvent mixtures with a higher polarity are not able to dissolve the preferred organic acids sufficiently and are therefore unsuitable.

The at least one hygroscopic solid substance of dispersion B in step b) according to the method of the invention comprises the hygroscopic solids already described in connection with the formulation according to the invention, which are further characterized in that for the method they are in the form of a powder.

Preferably the powder has an average particle size of 0.1-200 μm, especially preferably said powder has an average particle size of 0.1-20 μm.

Separation of the solid fraction from dispersion B, according to step c) of the method according to the invention, usually takes place by the methods for this that are generally known by a person skilled in the art, such as decanting, filtration, centrifugation etc.

The method according to the invention can be carried out with a step d) in the form of post-treatment, or without post-treatment. Preferably a post-treatment is carried out.

The post-treatment according to step d) of the method according to the invention usually comprises drying and/or washing of the solid fraction.

If washing is carried out, then the washing is preferably carried out together with a subsequent further separation according to step c) of the method according to the invention.

Usually the washing is carried out with a solvent for the acid used. Such solvents are for example water or alcohols or mixtures of water with alcohols.

It is especially advantageous to carry out washing, because in this way residues of the free acid can be removed from the solid fraction, so that the solid fraction is for example more suitable for use in pharmaceutical applications.

The drying can be carried out at ambient pressure (1013 HPa) or at reduced pressure relative to ambient pressure.

Furthermore, the drying is usually a thermal drying. Thermal drying means, in the context of the present invention, drying at temperatures that are increased, relative to normal conditions. Preferably, drying is carried out at temperatures that roughly correspond to the evaporation temperature of the solvent or solvent mixture 1, at the pressure during the drying. The evaporation temperatures of the solvents or solvent mixtures 1 at particular pressures are generally known by a person skilled in the art, for example from reference tables such as the VDI-Wärmeatlas.

In the case when washing with water has been carried out before drying, the temperature of thermal drying at ambient pressure (1013 HPa) is for example preferably 100° C.

The formulations according to the invention or the formulations according to the method of the invention are especially suitable for the use in connection with therapeutic procedures on mammals, preferably humans, or on domestic animals or pets.

Preferably the formulations according to the invention or the formulations according to the method of the invention find application as fillers or excipients of further formulations, which are used as medicinal products.

The invention is explained in more detail below with examples or on the basis of diagrams, though without being limited to these.

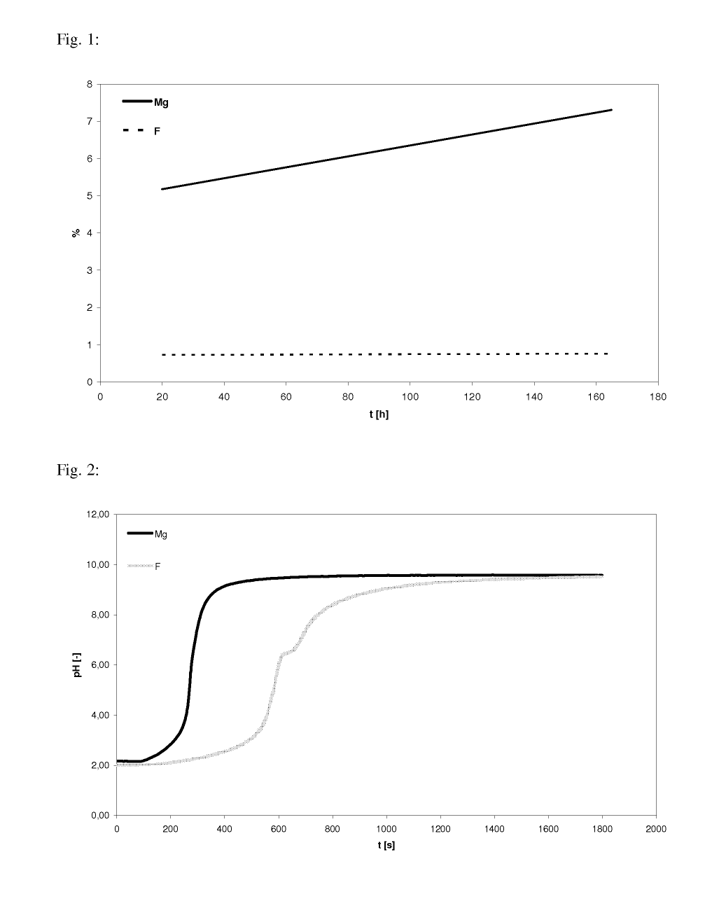

FIG. 1 shows the curve of the relative percentage weight change of a formulation according to the invention (F) and of pure magnesium hydroxide (Mg) when stored in air with 100% relative humidity and at 30° C.

FIG. 2 shows the variation of the pH of a formulation according to the invention (F) and of magnesium hydroxide (Mg), recorded according to example 3.

### EXAMPLES

### Example 1

### Production of a Formulation

25 g of pulverulent magnesium hydroxide was suspended in 135 g of n-hexane. This dispersion was heated to 30° C. Then 68 g tetradecanoic acid was dissolved in 90 g hexane. This solution was then fed as a continuous stream over a period of two minutes into the suspension already prepared. The resultant mixture was then stirred for four hours at room temperature.

The mixture was finally filtered with a paper filter and the solid obtained was washed with n-hexane. Finally the filter cake obtained was dried by open storage in the room air.

### Example 2

### Hygroscopicity

6.22 g of the dry filter cake from example 1, and 6.12 g of dry, pure magnesium hydroxide were in each case filled as a loose charge in a beaker. The beakers were then stored in a larger container, which was covered but not sealed, the bottom of which was covered with distilled water to a depth of 1 cm, at 30° C. in a drying cabinet. Any losses of water were made up sequentially, so that the free water surface remained the same throughout the test.

The weight change of the samples relative to their initial weight was measured. The results are presented in FIG. 1. It can be seen that, in contrast to the dry, pure magnesium hydroxide, the formulation according to the invention has a greatly reduced weight increase.

### Example 3

### Final pH of the Formulation

For verification of the final pH after dissolution, 100 g of 0.01-molar hydrochloric acid was put in a beaker and stirred with an inclined-vane stirrer at 400 rpm. Then 0.1 g of the solid to be verified (formulation from example 1, and magnesium hydroxide) was added and the increase in pH was recorded (pH-meter: Knick®Calimatic 766). The results are presented in FIG. 2. It can be seen that after a time of about 1800 s the same final pH is established in the case of pure magnesium hydroxide (Mg) and in the case of the formulation according to the invention (F). FIG. 2 also shows the delayed release of the magnesium hydroxide from the formulation according to the invention with almost the same gradient as in release of the pure magnesium hydroxide. The time delay was about 300 s.

1. Formulation of at least one solid substance that is hygroscopic under normal conditions, comprising:
a) a core, comprising the at least one hygroscopic solid substance and
b) a lipophilic shell surrounding the core,
where the core and the shell are bound together by ionic interaction.

2. Formulation according to claim 1, wherein the hygroscopic solid substance is an active substance for pharmaceutical use or an active substance for crop protection.

3. Formulation according to claim 1, wherein the hygroscopic solid substance is a filler, which together with active substances is permitted either for pharmaceutical use or for use in a plant protection agent.

4. Formulation according to claim 2, wherein the active substance is an oxide and/or hydroxide of alkaline-earth metals.

5. Formulation according to claim 1, wherein the lipophilic shell comprises a salt of the at least one hygroscopic solid substance with at least one organic acid.

6. Formulation according to claim 5, wherein the organic acid comprises a lipophilic moiety, which comprises at least four, non-ring carbon atoms and optionally comprises one or more multiple bonds between the at least four, non-ring carbon atoms.

7. Method of producing a formulation of at least one hygroscopic solid, which formulation comprises a core of at least one hygroscopic solid substance and a lipophilic shell, wherein said method comprises the steps:
c) preparing a solution A of at least one organic acid in a solvent or solvent mixture 1;
d) mixing solution A with a hygroscopic solid substance, obtaining a dispersion B;
e) separating the solid fraction from dispersion B;
f) optionally post-treating the separated solid fraction from dispersion B from c).

8. Method according to claim 7, wherein the solvent or solvent mixture 1 comprises nonpolar solvents or mixtures thereof with a polarity of max. 1.5 debye.

9. Method according to claim 7, wherein a post-treatment according to step d) is carried out.

10. Method according to claim 9, wherein the post-treatment comprises drying, where the drying is carried out substantially at temperatures that correspond to the evaporation temperature of the solvent or solvent mixture 1.

11. A method of treating a therapeutic condition in a mammalian patient in need of such treatment, comprising administering to said patient an amount effective to treat said therapeutic condition of a formulation according to claim 1.

12. (canceled)

13. Formulation according to claim 3, wherein the filler is an oxide and/or hydroxide of alkaline-earth metals.
